(19) United States
(12) Patent Application Publication
(10) Pub. No.: US 2007/0156165 A1
(43) Pub. Date: Jul. 5, 2007

(54) Percutaneous transgastric gastroplication and transgastric minimally invasive surgery

(76) Inventors: Kenneth J. Chang, Cerritos, CA (US); Ninh T. Nguyen, Ladera Ranch, CA (US)

Correspondence Address: BRINKS HOFER GILSON & LIONE/CHICAGO/COOK, PO BOX 10395, CHICAGO, IL, 60610, US

(21) Appl. No.: 11/489,248

(22) Filed: Jul. 19, 2006

Related U.S. Application Data
(60) Provisional application No. 60/701,645, filed on Jul. 21, 2005.

(51) Int. Cl. A61M 29/00

(52) U.S. Cl. 606/191

(57) ABSTRACT

Apparatus and methods for performing percutaneous transgastric or bi-transgastric minimally invasive surgical procedures targeting various organs and tissues of the gastrointestinal (GI) tract are provided. In one aspect of the invention, a percutaneous transgastric surgical port having at least one retraction tab disposed at a proximal end thereof is provided, wherein the at least one retraction tab is adapted for manipulating the percutaneous transgastric surgical port. In another aspect of the invention, a method of disposing one or more transgastric surgical ports in the gastric wall of a patient is provided, the one or more ports being utilized to perform a transgastric or bi-transgastric procedure in which the stomach itself may serve as a surgical port.

### RELATED APPLICATION

This application claims the benefit of priority from U.S. Provisional Application No. 60/701,645 filed Jul. 21, 2005, and entitled “Percutaneous Transgastric Gastroplication and Transgastric Minimally Invasive Surgery”, which is incorporated herein by reference.

### TECHNICAL FIELD

The present invention relates to medical devices and procedures, and more particularly to apparatus and methods for performing percutaneous transgastric and bi-transgastric minimally invasive surgical procedures targeting various organs and tissues of the gastrointestinal (GI) tract.

### BACKGROUND OF THE INVENTION

Conventional intra-abdominal surgery has typically been performed by forming an incision in the abdominal wall of the patient. The incision is then used to access and perform operations on the internal body organs located within the patient's abdominal cavity. This method of surgery invariably results in substantial blood loss, as well as considerable discomfort or pain to the patient after the surgery has been completed.

The development of laparoscopic surgery techniques has reduced many of the disadvantages of conventional intra-abdominal surgery. In laparoscopic surgery, the abdominal organs are accessed via trocar sleeves and/or laparoscopic cannulas disposed in perforations formed in the abdominal wall of the patient by trocars. The sizes of the perforations formed by the trocars are much smaller than the incision typically required for conventional surgery. As a result, patient discomfort and complications, as well as hospital duration, are greatly reduced.

However, it is often difficult to access and perform operations on certain bodily organs using laparoscopic surgery techniques. For example, it is often difficult to access and operate on the pancreas because of its proximity to the gastric lumen (stomach). In addition, certain medical procedures may require simultaneous access to various abdominal organs within the abdominal cavity and the internal passageways of the gastrointestinal (GI) tract. Ordinarily, the internal passageways of the GI tract are accessed via an endoscope introduced through the patient's esophagus or colon. However, the manipulation of an endoscope and/or endoscopic devices may be cumbersome or difficult while simultaneously performing other procedures using laparoscopic techniques. Moreover, endoscopic access to the GI tract via natural passageways may not be desirable or possible in certain patients because of the presence of disease or obstructions within these natural passageways.

### SUMMARY OF THE INVENTION

Broadly, the present invention provides apparatus and methods for performing percutaneous transgastric or bi-transgastric minimally invasive surgical procedures targeting various organs and tissues of the gastrointestinal (GI) tract. In one embodiment, the present invention may provide apparatus comprising a percutaneous transgastric surgical port and at least one retraction tab disposed at a proximal end of the surgical port, wherein the at least one retraction tab may be adapted for manipulating the percutaneous transgastric surgical port. The apparatus of the present invention may further comprise a gas supply conduit and gas supply valve adapted for insufflation, via the percutaneous transgastric surgical port, of a body cavity such as the stomach or the peritoneum. As a non-limiting example, the present invention may be used for performing percutaneous transgastric gastroplication, e.g., for the treatment of gastroesophageal reflux disease (GERD). The present invention may also be used for resection of lesions inside the GI tract (mucosal and submucosal), and for performing bi-transgastric procedures in which the stomach itself may serve as a surgical port. Such procedures performed according to the present invention may include, without limitation, cholecystectomy, appendectomy, tubal ligation, choledochoduodenostomy, choledochogastrostomy, and the like.

In contrast to the present invention, gastrostomy tubes and laparoscopic ports of the prior art lack both a gas supply conduit and an external bumper having at least one retraction tab. In contrast to prior art procedures, the present invention may provide for the creation of an extension of the esophagus by gastroplication at the gastroesophageal junction (GEJ), wherein the extension of the esophagus may be in the form of a “pseudo-esophagus” located within the stomach at a location inferior to the GEJ. Also in contrast to prior art procedures, the present invention may provide, in another embodiment, GI tract procedures in which surgical instruments are introduced into the stomach via a percutaneous transgastric surgical port (PTSP), and the instruments are subsequently introduced to a target site of the GI tract via the stomach.

These and other advantages, as well as the invention itself, will become apparent in the details of construction and operation as more fully described below. Moreover, it should be appreciated that several aspects of the invention can be used with other types of stent delivery catheters or medical devices.

### BRIEF DESCRIPTION OF THE DRAWINGS

FIG. 1 is a sectional view of an apparatus for providing percutaneous access to the stomach, including a percutaneous transgastric surgical port having an attached dilating portion, according to one aspect of the present invention;

FIG. 2 is a sectional view of a percutaneous transgastric surgical port in situ, according to an embodiment of the present invention;

FIG. 3 is an end view of the proximal end of a percutaneous transgastric surgical port having an external bumper thereon, according to the invention;

FIG. 4 is a sectional view of a percutaneous transgastric surgical port having a gas supply conduit for passage of insufflation gas, according to another embodiment of the present invention;

FIG. 5 schematically represents a series of steps involved in a method for performing a percutaneous transgastric procedure, according to another embodiment of the invention;

FIGS. 6A and 6B schematically represent the stomach of a patient prior to gastroplication and after gastroplication, respectively, according to another embodiment of the invention; and

FIG. 7 schematically represents a series of steps involved in a method of treating gastroesophageal reflux disease, according to another embodiment of the invention.

### DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

FIG. 1 is a sectional view of a percutaneous surgical access apparatus (PSAA) 10 for providing percutaneous surgical access to the stomach of a patient, according to one aspect of the present invention. Apparatus 10 may include a percutaneous transgastric surgical port (PTSP) 20 having a port proximal end 20a and a port distal end 20b. PTSP 20 may be adapted for direct percutaneous surgical access to the interior of the stomach of a patient, and PTSP 20 may span both the gastric wall and the abdominal wall when PTSP 20 is in situ. PTSP 20 may comprise a plastic or metal tube, and may have an internal diameter typically from about 5 to 15 mm. PTSP 20 may have a length typically in the range of from about 2 to 4 cm, and often from about 2.5 to 3 cm. A valve or seal (not shown) may be disposed within PTSP 20 to allow the passage of instruments through PTSP 20 without the release of insufflation gas from the patient's stomach.

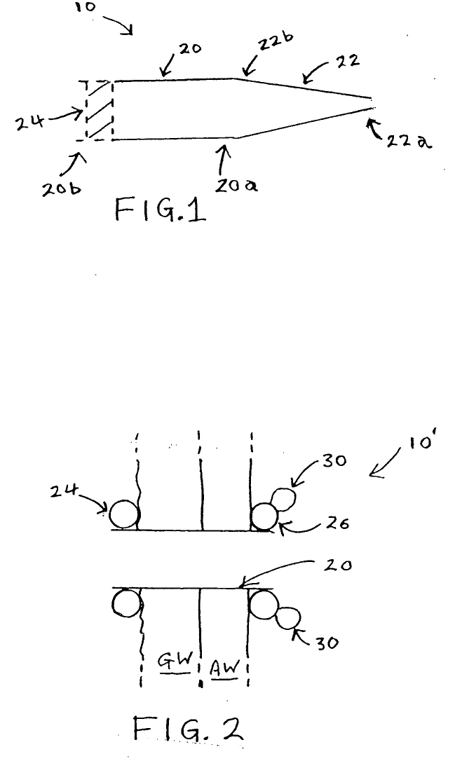

Apparatus 10 may further include a dilating portion 22 disposed at port proximal end 20a. Dilating portion 22 may include a dilating portion proximal end 22a and a dilating portion distal end 22b. Dilating portion 22 may taper from broad to narrow in a proximal direction, i.e., from dilating portion distal end 22a to dilating portion proximal end 22b. Dilating portion 22 may be adapted for dilating an incision or hole formed in the gastric wall and abdominal wall of a patient (see, for example, FIG. 2). Dilating portion 22 may be further adapted for being removed from port proximal end 20a, for example, after PTSP 20 has been suitably placed transgastrically in the stomach of a patient. Dilating portion 22 may be adapted for being detached from port proximal end 20a, for example, via a bayonet fit. Alternatively, dilating portion 22 may be simply severed or cut from port proximal end 20a. PTSP 20 may include an internal bumper 24 disposed at port distal end 20b. Internal bumper 24 may be inflatable (see, for example, FIG. 2). FIG. 1 shows internal bumper 24 in the uninflated state.

FIG. 2 is a sectional view of a percutaneous surgical access apparatus 10′, placed in situ, for providing percutaneous surgical access to the stomach of a patient, according to an embodiment of the present invention. Apparatus 10′ may include percutaneous transgastric surgical port (PTSP) 20. As shown, PTSP 20 may extend through both the gastric wall GW and the abdominal wall AW. (The skin and peritoneum are omitted from FIG. 2 for the sake of clarity.) Internal bumper 24 may be inflated so as to retain port distal end 20b in position with respect to the gastric wall GW and to prevent axial movement of PTSP 20 in a proximal direction.

Apparatus 10′ may further include an external bumper 26 disposed at port proximal end 20a. External bumper 26 may be adapted for retaining port proximal end 20a in position with respect to the abdominal wall AW and to prevent axial movement of PTSP 20 in a distal direction. External bumper 26 may be placed on port proximal end 20a by a member of the surgical team, e.g., after internal bumper 24 has been inflated. External bumper 26 may comprise a resilient material, such as various rubber compositions.

Apparatus 10′ may further include at least one retraction tab 30. Retraction tabs 30 may be disposed adjacent to port proximal end 20a. As a non-limiting example, retraction tabs 30 may be disposed on external bumper 26. Retraction tabs 30 may comprise a resilient material, such as various compositions of rubber or plastics. In addition, retraction tabs 30 are specifically configured to permit grasping and manipulation by a user. More specifically, retraction tabs 30 may be used for grasping by a member of the surgical team for manipulating PTSP 20. As an example, retraction tabs 30 may be used to decrease tension exerted by PTSP 20 on abdominal wall AW during passage of instruments through PTSP 20. A reduction in tension may help to reduce patient discomfort and prevent irritations and/or infections from developing in the tissue surrounding the PTSP. Retraction tabs 30 may also be used to aim or point PTSP 20 in a particular direction, for example, in the direction of a particular target tissue. Changing the direction of PTSP 20 with respect to a target tissue, may decrease the amount of instrument steering required during a particular transgastric procedure.

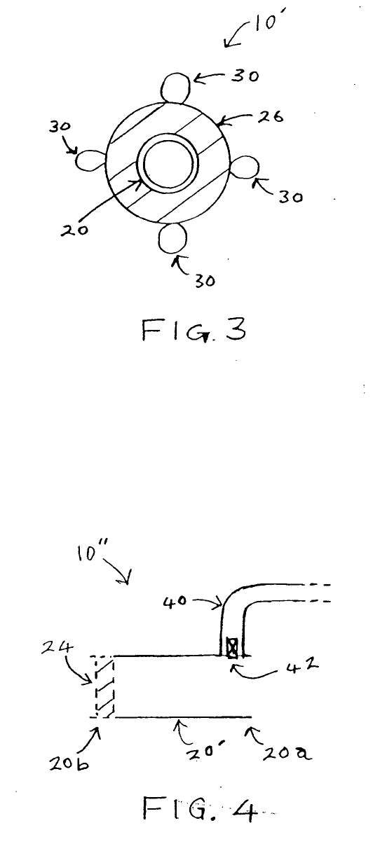

FIG. 3 is an end view of apparatus 10′ showing the proximal end of PTSP 20 having an external bumper 26 disposed thereon, according to an embodiment of the present invention. As shown, a plurality of retraction tabs 30 may be circumferentially spaced on external bumper 26, such that one or more of retraction tabs 30 may be conveniently grasped during a percutaneous transgastric procedure for manipulation of PTSP 20. In some embodiments, retraction tabs 30 may be integral with external bumper 26. Although four retraction tabs 30 are shown in FIG. 3, other numbers of retraction tabs 30 are also within the scope of the present invention. Typically, apparatus 10′ may comprise from 1 to about 4 retraction tabs 30, and often from about 2 to 4 retraction tabs 30.

FIG. 4 is a sectional view of a percutaneous surgical access apparatus 10″, according to another embodiment of the present invention. Apparatus 10″ may include a percutaneous transgastric surgical port (PTSP) 20′. PTSP 20′ may be adapted for passage therethrough of insufflation gas, for example, for insufflation of the stomach or the peritoneum. PTSP 20′ may have a port proximal end 20a, a port distal end 20b, and an internal bumper 24 at, or adjacent to, port distal end 20b.

Apparatus 10″ may further include a gas supply conduit 40. PTSP 20′ may be adapted for coupling to gas supply conduit 40 at port proximal end 20a. Gas supply conduit 40 may be configured or adapted for coupling to a gas supply unit (not shown) for providing insufflation gas to PTSP 20′. Gas supply units for providing a supply of insufflation gas, such as air or carbon dioxide, at a pre-determined pressure, are well known in the art. PTSP 20′ may further include a gas supply valve 42 for controlling the flow of insufflation gas through PTSP 20′. Gas supply valve 42 may be housed within gas supply conduit 40. PTSP 20′ may have additional features, for example, as described for PTSP 20 with reference to FIGS. 1-3.

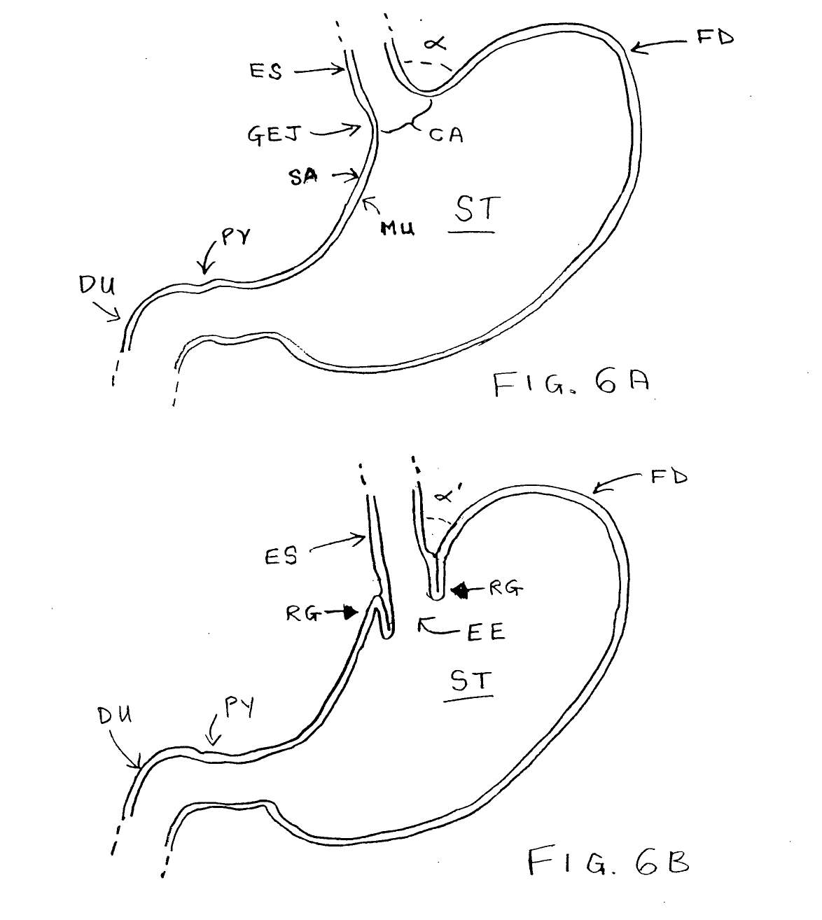

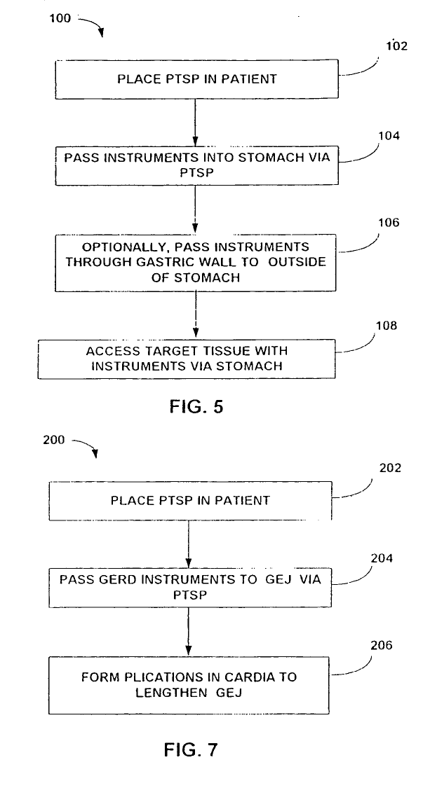

FIG. 5 schematically represents a series of steps involved in a method 100 for performing a percutaneous minimally invasive transgastric surgical procedure on a patient, according to another embodiment of the invention. Method 100 may include a step 102 of placing or installing a percutaneous transgastric surgical port (PTSP) to provide percutaneous access to the interior of the stomach of the patient. The PTSP may be a part of a percutaneous surgical access apparatus (PSAA), such as apparatus 10 (FIG. 1). The PSAA and PTSP may have elements and features as described hereinabove with reference to FIG. 1-4.

The PTSP may be placed through both the gastric wall and the abdominal wall (see, for example, FIG. 2). As an example, the PTSP may be placed in the patient by a process involving: insufflation of the stomach via an endoscope, such that the gastric wall is disposed against the abdominal wall; percutaneously introducing a needle through an incision in the abdominal wall into the insufflated stomach; passing a guidewire through the needle into the stomach; grasping the distal end of the guidewire from within the stomach, via the endoscope, and retracting the guidewire such that the distal end of the guidewire exits the mouth of the patient; passing the PSAA over the guidewire through the mouth and into the stomach; passing the dilating portion of the PSAA through the incision in the abdominal wall such that the dilating portion protrudes from the abdominal wall; inflating an internal bumper of the PTSP; pulling the dilating portion in a direction away from the abdominal wall until the internal bumper contacts the interior of the gastric wall; clasping an external bumper on the proximal end of the PTSP; and removing or detaching the dilating portion of the PSAA to provide the PTSP in situ. Accordingly, the PTSP may be fixed in position with respect to both the gastric wall and the abdominal wall via the internal bumper and the external bumper (see, for example, FIG. 2).

One or more retractor tabs may be disposed at the proximal end of the PTSP. As an example, the one or more retractor tabs may be integral with the external bumper. Typically, the PTSP may comprise from one (1) to about four (4) retractor tabs.

Again with reference to FIG. 5, step 104 may involve passing surgical instruments into the stomach via the PTSP. During the passage of instruments into and out of the stomach via the PTSP, the PTSP may be grasped via the one or more retractor tabs in order to decrease the amount of tension applied to the abdominal wall. Instruments passed into the stomach via the PTSP in step 104 may be adapted for percutaneous transgastric or bi-transgastric procedures. Instruments passed into the stomach via the PTSP may include, without limitation, suturing, cautery, ligation, and knot-tying devices. Such instruments are generally known in the art.

Depending on the location of the target tissue or organ for a particular procedure, the target tissue may be accessed with the surgical instruments (step 108) via the stomach in various ways. As an example, the stomach itself may be accessed directly for procedures such as gastroplication (see, FIGS. 6A-B, 7). According to another aspect of the present invention, target sites of the GI tract adjacent to the stomach, e.g., the duodenum and esophagus, may be accessed via the stomach by guiding the instruments internally within the GI tract.

According to yet another aspect of the present invention, access to certain target sites may require step 106, which may involve a second transgastric manipulation of the surgical instruments (wherein passage of the instruments into the stomach via the PTSP in step 102 may comprise a first transgastric manipulation of the surgical instruments). More specifically, a second PTSP of the type described above may be placed through the gastric wall at a location that is spaced apart from the location of the first PTSP (which extends simultaneously through both the gastric and abdominal wall). Preferably, the second PTSP is located in a wall of the stomach that is opposite to the wall of the stomach in which the first PTSP is located. The second PTSP provides access to the abdominal cavity from within the stomach. Thus, in step 106 the instruments may be passed from within the stomach through the gastric wall of the patient to a location outside the stomach towards the target site. More specifically, the instruments are passed into the stomach via the first PTSP, then across the interior volume of the stomach, and subsequently out of the stomach and into the abdominal cavity via the second PTSP. Accordingly, procedures which include step 106 may be referred to as “bi-transgastric.” A bi-transgastric procedure may be particularly advantageous in accessing and operating on organs that may otherwise be difficult to access utilizing laparoscopic surgery techniques. For example, a bi-transgastric procedure facilitates easy access to the pancreas, which is often difficult to reach because of its proximity to the stomach.

The second PTSP may be placed utilizing a similar procedure as described above for placing the first PTSP. For example, with the aid of an endoscope, an incision is made in the gastric wall at the desired location. A guidewire is then passed through the through the mouth of the patient, into the stomach, and through the incision. The second PSAA is then passed over the guidewire through the mouth and into the stomach. The distal portion of the second PSAA is then passed through the incision in the gastric wall such that the distal portion extends outside the stomach and into the abdominal cavity. The internal and external bumpers of the second PTSP are then inflated so as to secure the second PTSP in situ. Alternatively, the second PTSP may be placed via the first PTSP, thereby eliminating the need to introduce the second PTSP via the patient's esophagus.

It should be noted that placement of the second PTSP is much more easily accomplished once the first PTSP has been placed through the gastric and abdominal wall of the patient. This is because the first PTSP serves to “anchor” or otherwise secure the stomach to the abdominal wall. In addition, and as discussed above, the first PTSP can be utilized to facilitate the placement of the second PTSP.

Preferably, the second PTSP is more flexible in its construction than that of the first PTSP. The increased flexibility of the second PTSP permits greater manipulation of any surgical tools passing therethrough, and may tend to form a better seal with the gastric wall. The first PTSP, in contrast, must have greater rigidity (as compared to the second PTSP) because of its placement through the abdominal wall of the patient.

As a non-limiting example, a bi-transgastric procedure of the present invention may involve a choledochoduodenostomy, in which a communication may be formed between the common bile duct and the duodenum. Other procedures that may be performed via the percutaneous transgastric route according to the invention include, without limitation, cholecystectomy, appendectomy, tubal ligation, and choledocho-gastrostomy. Typically, the target tissue accessed via the stomach in steps 106 and 108 may be located adjacent to, or within, the GI tract.

In addition to the placement of a first and second PTSP's in the gastric wall of the patient, a third PTSP may be placed through the abdominal wall of the patient. Unlike the first and second PTSP's, the third PTSP does not engage or extend through the gastric wall of the patient. The third PTSP can be utilized in combination with the first and second PTSP's to perform various diagnostic or surgical procedures.

After the target tissue has been accessed (step 108), T-bars (not shown) may be placed on each side of the PTSP, prior to removing the PTSP, to prevent the gastric wall from moving away from the abdominal wall. After placement of the T-bars and removal of the PTSP, the wound may be closed, e.g., with 1 or 2 sutures.

FIGS. 6A and 6B schematically represent the stomach ST of a patient prior to gastroplication and after gastroplication, respectively, according to another embodiment of the invention. With reference to FIG. 6A, the stomach ST includes the cardia CA (or lower esophageal sphincter) and the fundus FD disposed superior to the gastroesophageal junction (GEJ). The stomach ST includes the inner mucosa MU and the outer serosa SA. The duodenum DU exits the stomach ST at the pylorus PY. The esophagus ES may join the stomach ST in the region of the GEJ to define an angle of His, α. The angle of His, α is an acute angle formed between the cardia of the stomach and the distal esophagus ES, and functions as a flap at the GEJ to help prevent reflux of gastric contents into the esophagus. Nevertheless, the gastroesophageal junction may represent an incomplete barrier to the reflux of gastric contents into the esophagus, hence the need for treatments such as gastroplication according to the instant invention.

With reference to FIG. 6B, and according to one embodiment of the present invention, a PTSP 20/20′ (hereafter PTSP 20; see, for example, FIGS. 2-4) may be introduced percutaneously into the stomach ST of the patient. Instrumentation may be introduced into the stomach, via PTSP 20, for gastroplication in the region of the GEJ such that the esophagus is effectively extended distally into the stomach to form an esophagus extension EE, or “pseudo-esophagus.” At the same time, gastroplication in the region of the GEJ may result in a decrease in the angle of His, from angle α (FIG. 6A) to angle α′ (FIG. 6B), i.e., the angle of His may be made more acute as a result of gastroplication according to the invention. A region of gastroplication RG according to one embodiment of the invention may be indicated in FIG. 6B by the solid arrows. Although the plications are shown in FIG. 6B as being serosa to serosa, other plication configurations, such as mucosa to mucosa, are also within the scope of the invention. A method for treating GERD via gastroplication according to the instant invention is described hereinbelow.

FIG. 7 schematically represents a series of steps involved in a method 200 for treating GERD in a patient, according to another embodiment of the invention. Method 200 may involve a step 202 of placing or installing a percutaneous transgastric surgical port (PTSP) to provide percutaneous access to the interior of the stomach of the patient. Step 202 may be performed substantially as described hereinabove with reference to FIG. 5 for step 102 of method 100. Thereafter, step 204 may involve the passage of instrumentation for the treatment of GERD, which may include suturing, cautery, ligation, and knot-tying devices, through the PTSP into the region of the gastroesophageal junction (GEJ) and proximal stomach.

Thereafter, step 206 may involve forming plications in the cardia of the stomach. The plications may result in lengthening of the GEJ and the formation of an extension of the esophagus (see, FIG. 6B). The extension of the esophagus may be formed internally within the stomach at a location inferior to the GEJ. The extension of the esophagus may create a “pseudo-esophagus” in communication with the esophagus. The extension of the esophagus may be formed by a plurality of plications in the region of the GEJ. Typically, about 4 to 12 plications, and often about 7 to 10 plications, may be placed in the region of the GEJ.

The plications may be made with sutures in the cardia of the stomach. In one embodiment, the plications may be made by adjoining serosa to serosa, i.e., with the serosa of the stomach in apposition. Pledgets may be placed to decrease suture tears. Plications may be secured using either traditional knots or knot-tying devices. The plications thus formed may result in tightening of the GEJ, and the creation of a “wind-sock” or tunnel effect, which may actually lengthen the GEJ and reduce reflux of gastric contents into the esophagus. During such gastroplication according to the present invention, the angle of His may also be made more acute; for example, the angle of His may be decreased from angle α to angle α′ (see, FIG. 6B), where α>α′.

Although the invention has been described primarily in relation to gastroplication and the treatment of GERD, the present invention may also be used for numerous other procedures and for treating organs other than the stomach.

It should be understood, of course, that the foregoing relates to exemplary embodiments of the invention and that modifications may be made without departing from the spirit and scope of the invention as set forth in the following claims.

1. Apparatus for percutaneous surgical access to the stomach of a patient, comprising:
a percutaneous transgastric surgical port having a proximal end and a distal end, said percutaneous transgastric surgical port adapted for the passage of surgical instruments therethrough into the stomach of the patient;
an external bumper disposed on said proximal end, said external bumper adapted for restricting distal axial movement of said percutaneous transgastric surgical port;
an internal bumper disposed on said distal end, said internal bumper adapted for restricting proximal axial movement of said percutaneous transgastric surgical port; and
at least one retraction tab disposed on said external bumper, the at least one retraction tab adapted for manually manipulating said percutaneous transgastric surgical port with respect to the abdominal wall of the patient.

2. The apparatus according to claim 1 further comprising a dilating portion detachable from said port proximal end.

3. The apparatus according to claim 1 further comprising a gas supply conduit disposed at said port proximal end, wherein said percutaneous transgastric surgical port is adapted for insufflation of at least one of the stomach and the peritoneum of the patient.

4. A system for percutaneous surgical bi-transgastric access to the peritoneum of a patient, comprising:
a first transgastric surgical port having a proximal end, a distal end, an external bumper disposed on said proximal end, and an internal bumper disposed on said distal end, said first transgastric surgical port being percutaneously disposed through both an abdominal wall and a gastric wall of the patient and adapted for the passage of surgical instruments therethrough into the stomach of the patient; and
a second transgastric surgical port having a proximal end, a distal end, an external bumper disposed on said proximal end, and an internal bumper disposed on said distal end, said second transgastric surgical port being disposed only through the gastric wall of the patient at a location spaced apart from the first transgastric surgical port, the second transgastric surgical port being adapted for the passage of surgical instruments therethrough out of the stomach and into the peritoneum.

5. The system according to claim 4 further comprising surgical instruments simultaneously disposed through the first transgastric surgical port and the second transgastric surgical port.

6. The system according to claim 5 wherein the surgical instruments traverse the interior volume of the stomach.

7. The system according to claim 4 wherein the first transgastric surgical port and the second transgastric surgical port are disposed in opposing gastric walls of the stomach.

8. A method for performing a bi-transgastric procedure on a patient, comprising:
a) placing a first transgastric surgical port percutaneously through both an abdominal and a gastric wall of the patient;
b) placing a second transgastric surgical port through the gastric wall of the patient at a location spaced apart from the first transgastric surgical port;
c) thereafter, passing surgical instruments percutaneously into the stomach via said first transgastric surgical port in a first transgastric manipulation of said surgical instruments;
d) thereafter, passing said surgical instruments from within the stomach through the gastric wall of the patient to a location outside the stomach via said second transgastric surgical port in a second transgastric manipulation of said surgical instruments; and
e) thereafter, accessing a target tissue within the peritoneum of the patient with said surgical instruments.

9. The method according to claim 8 wherein the first transgastric surgical port comprises at least one retraction tab disposed on external portion thereof, the method further comprising the step of manually manipulating the at least one retraction tab so as to manipulate the position of said first transgastric surgical port with respect to the abdominal wall of the patient.

10. The method according to claim 8 wherein the first transgastric surgical port comprises a gas supply conduit disposed thereon, the method further comprising the step of passing CO₂ through the gas supply conduit so as to insufflate at least one of the stomach and the peritoneum of the patient.

11. A method for treating gastroesophageal reflux disease in a patient, comprising:
a) placing a percutaneous transgastric surgical port through the gastric wall of the patient;
b) thereafter, percutaneously passing surgical instruments for the treatment of said gastroesophageal reflux disease through said percutaneous transgastric surgical port into the region of the gastroesophageal junction of the stomach of the patient; and
c) thereafter, forming plications in the cardia of the stomach so as to form an extension of the esophagus within the stomach and so as to lengthen the gastroesophageal junction.
